(19) United States
(12) Patent Application Publication
(10) Pub. No.: US 2008/0125785 A1
(43) Pub. Date: May 29, 2008

(54) Targeting Device

(76) Inventor: Gursharan Singh Chana, Sutton Coldfield (GB)

Correspondence Address: ICE MILLER LLP, ONE AMERICAN SQUARE, SUITE 3100, INDIANAPOLIS, IN, 46282-0200, US

(21) Appl. No.: 11/535,051

(22) Filed: Sep. 25, 2006

(51) Int. Cl. A61B 17/56

(52) U.S. Cl. 606/96

(57) ABSTRACT

A targeting device for use in hip surgery and more particularly for use in hip replacement surgery or hip resurfacing surgery, which may include minimally invasive surgery. An exemplary embodiment of the targeting device comprises one or more sensor devices, a guide sleeve, a guide sleeve location adjustor, and a computer device with software for receiving data from the sensor device or devices and processing this data so as to determine the location of the centerline of the femoral neck. In other embodiments, the sensor devices are movable so as to obtain data at two or more different radial locations around the femoral neck or angularly spaced in order to obtain data in the anterior-posterior plane and in the superior-inferior plane. Moreover, the sensor devices can be placed on retractable arms.

### PRIORITY

The present application claims priority of U.K. Patent Application No. ______, filed Sep. 19, 2006.

### BACKGROUND

The present disclosure relates to a targeting device, particularly for use in hip surgery and more particularly for use in hip replacement surgery or hip resurfacing surgery. The disclosure also relates to the use of the device in surgery, particularly minimally invasive surgery.

When performing hip replacement surgery it is desired to be able to accurately determine where the center of the base of the femoral neck and the centerline through the femoral neck lie to allow correct reaming fitting of the replacement head.

Equally, when performing hip resurfacing surgery it is desired to be able to accurately determine where the centerline through the femoral neck and femoral head lies to allow correct reaming of the femoral head and correct fitting of the prosthesis onto the femoral head.

In the event that the centerline is not accurately determined, there is a high risk of femoral neck fracture and failure of the implant, thus leading to a need for revision surgery. As well as being a secondary operation this can be quite distressing for the patient and is also a more expensive procedure.

The position of the femoral head does not always assist in determining the position of the center of the femoral neck and the centerline through the femoral neck as the head may not be centrally positioned in relation to the femoral neck due to deformity of the femoral head.

Accordingly there is the need for a targeting device which is suitable to allow the position of the center of the femoral neck and/or the centerline through the femoral neck and femoral head (hereinafter reference to “the centerline of the femoral neck” is used to cover both of these references) to be determined, which device can be used in minimally invasive surgical methods and in conventional open surgical methods.

### BRIEF DESCRIPTION

In an exemplary embodiment, there is provided a targeting device for use in hip surgery to allow the position of the centerline of the femoral neck to be located, the device comprising one or more sensor devices, moveable to a position inside the body that is over the femoral neck and radially spaced therefrom, the device or devices being suitable for obtaining data relating to the location of the surface of the femoral neck with respect to a reference position, in both the anterior-posterior and superior-inferior planes; a guide sleeve, having a bore capable of receiving a guide wire; a guide sleeve location adjustor, suitable for moving the guide sleeve in the anterior, posterior, medial and lateral directions; and a computer device with software for receiving data from the sensor device or devices and processing this data so as to determine the location of the centerline of the femoral neck.

In an additional embodiment, there may be one or more sensor devices that are moveable so as to obtain data at two or more different radial locations around the femoral neck, with at least one location being such that data is obtained in the anterior-posterior plane and at least one location being such that data is obtained in the superior-inferior plane.

The one or more sensor devices may be moveable so as to obtain data at four or more different radial locations around the femoral neck. In yet another embodiment, the one or more sensor devices are moveable to two or more different radial locations where data is obtained in the superior-inferior plane. In an additional embodiment, the one or more sensor devices are moveable to two or more different radial locations where data is obtained in the anterior-posterior plane.

The targeting device may comprise two or more angularly spaced sensor devices with at least one device being able to obtain data in the anterior-posterior plane and at least one device being able to obtain data in the superior-inferior plane. In another embodiment, the targeting device may have four or more such angularly spaced sensor devices. The angular devices can comprise two or more devices able to obtain data in the superior-inferior plane and/or two or more devices able to obtain data in the anterior-posterior plane.

The sensor devices of the targeting system can be selected from electro-mechanical devices, radar devices, infrared devices, ultrasound devices and electromagnetic devices. It will be appreciated by one skilled in the art the other suitable devices may also be used.

In another embodiment, the targeting device may comprise one or more retractable arms, each of which can move from a retracted state to an extended state. The one or more sensor devices can be located on the one or more retractable arms. Preferably, there are the same number of arms as sensor devices, and a single sensor device is provided on each arm. In an exemplary embodiment, the one or more retractable arms are a curved shape. In another exemplary embodiment, the one or more retractable arms are rigid.

The targeting device may comprise a number of other components, including a sleeve component. The sleeve component can comprise a first wall having an exterior surface and an interior surface that defines an inner bore and runs from a proximal end of the sleeve component to a distal end of the sleeve component. In another embodiment, the first wall of the sleeve component forms a cylinder shape. The sleeve component can be shaped and sized such that it can be placed over the femoral neck with some or all of the femoral neck located within the bore. In an exemplary embodiment of the targeting device, the one or more sensor devices are located in the sleeve component.

The targeting device can be used in a method of locating the centerline of the osteotomised base of the femoral neck at the head-neck junction. The method can comprise the steps of providing a targeting device with one or more sensor devices that are moveable to a position inside the body that is over the femoral neck and radially spaced therefrom. The sensor device or devices are suitable for obtaining data relating to the location of the surface of the femoral neck with respect to a reference position, in both the anterior-posterior and superior-inferior planes. The targeting device also can comprise a guide sleeve, having a bore capable of receiving a guide wire; a guide sleeve location adjustor, suitable for moving the guide sleeve in the anterior, posterior, medial and lateral directions; and a computer device with software for receiving data from the sensor device or devices and processing this data so as to determine the location of the centerline of the femoral neck. The method also comprises the step of moving the or each sensor device to a position inside the body, over the femoral neck and radially spaced therefrom and using the or each sensor device to obtain data relating to the distance between the sensor device and the surface of the femoral neck in at least one location in the anterior-posterior plane and at least one location in the superior-inferior plane. This method of using the target device also comprises the step of using the software to determine the position of the centerline of the femoral neck based on the data received from the or each sensor device and using the guide sleeve location adjustor to move the guide sleeve to be in line with the determined centerline.

The targeting devices may also be used in a method of locating the centerline through the femoral neck and femoral head comprising of the same steps as described above.

### BRIEF DESCRIPTION OF THE DRAWINGS

The features and advantages of this disclosure, and the manner of attaining it, will be more apparent and better understood by reference to the following descriptions taken in conjunction with the accompanying drawings, wherein:

FIG. 1 shows a perspective view of an exemplary targeting device;

FIG. 2 shows a front view of the targeting device of FIG. 1;

FIG. 3 shows a cross section through line B-B of the targeting device of FIG. 2;

FIG. 4 shows a perspective view of another exemplary targeting device;

FIG. 5 shows a side view of the targeting device of FIG. 4; and

FIG. 6 shows a front view of the targeting device of FIG. 4.

### DETAILED DESCRIPTION

In an exemplary embodiment, there is provided a targeting device for use in hip surgery to allow the position of the centerline of the femoral neck to be located. The targeting device may comprise one or more sensor device that are moveable to a position inside the body that is over the femoral neck and radially spaced therefrom and that are suitable for obtaining data relating to the location of the surface of the femoral neck with respect to a reference position, in both the anterior-posterior and superior-inferior planes. The targeting device may also comprise a guide sleeve, having a bore capable of receiving a guide wire; a guide sleeve location adjustor, suitable for moving the guide sleeve in the anterior, posterior, medial and lateral directions; and a computer device with software for receiving data from the sensor device or devices and processing this data so as to determine the location of the centerline of the femoral neck.

The one or more sensor devices may be any device or combination of devices that permits direct or indirect measurement of distance, in both the anterior-posterior and superior-inferior planes, between a reference location and the surface of the femoral neck when the one or more sensor devices are located at a position or positions inside the body, over the femoral neck and radially spaced therefrom.

In an additional embodiment, there may be one or more sensor devices that are moveable so as to obtain data at two or more different radial locations around the femoral neck, with at least one location being such that data is obtained in the anterior-posterior plane and at least one location being such that data is obtained in the superior-inferior plane.

In an additional embodiment, the one or more sensor devices are moveable so as to obtain data at three or more different radial locations around the femoral neck or more preferably four or more different radial locations around the femoral neck (e.g., six or more different radial locations around the femoral neck).

In an additional embodiment, the one or more sensor devices are moveable to two or more different radial locations where data is obtained in the superior-inferior plane (e.g., three or more different radial locations where data is obtained in the superior-inferior plane).

In an additional embodiment, the one or more sensor devices are moveable to two or more different radial locations where data is obtained in the anterior-posterior plane (e.g., three or more different radial locations where data is obtained in the anterior-posterior plane).

In an exemplary embodiment, there may be two or more angularly spaced sensor devices, which may be fixed or moveable, with at least one device being able to obtain data in the anterior-posterior plane and at least one device being able to obtain data in the superior-inferior plane.

In an additional embodiment, there are three or more angularly spaced sensor devices. More preferably, there are four or more angularly spaced sensor devices (e.g., six or more angularly spaced sensor devices). For example, there may be four angularly spaced sensor devices, spaced at angles of 90° apart.

In an additional embodiment, there are two or more devices able to obtain data in the superior-inferior plane, such as three or more devices able to obtain data in the superior-inferior plane, for example four or more devices able to obtain data in the superior-inferior plane.

In an additional embodiment, there are two or more devices able to obtain data in the anterior-posterior plane, such as three or more devices able to obtain data in the anterior-posterior plane, for example four or more devices able to obtain data in the anterior-posterior plane.

The sensor devices may suitably be selected from electromechanical devices, radar devices, infrared devices, ultrasound devices and electromagnetic devices. For example, electromechanical devices can be used to obtain data relating to the distance between a reference location and the surface of the femoral neck. Specifically, the or each electromechanical device may be arranged so as to be able to move from a neutral position, located radially with respect to the femoral neck, to a position abutting the surface of the femoral neck. The or each electromechanical device is arranged to be able to record the distance moved by the electromechanical device from the neutral position to the position abutting the surface of the femoral neck. Accordingly, the reference location is the neutral position of the electromechanical device.

In an additional embodiment, the or each electromechanical device is be able to move in a straight line from the neutral position to the position abutting the surface of the femoral neck. In order to accomplish this movement, the or each sensor device may comprise an electronically driven motor that is be able to move in a straight line from the neutral position to the position abutting the surface of the femoral neck. It is also desirable for the or each electromechanical device to be able to move back from the position abutting the surface of the femoral neck to the neutral position. The distance travelled back from the position abutting the surface of the femoral neck to the neutral position may also be recordable by the or each sensor device.

Radar devices can be used to obtain data relating to the distance between a reference location and the surface of the femoral neck. Specifically, the or each radar device may be arranged so as to be able to emit a radar beam from a radial position with respect to the femoral neck, towards the femoral neck. The radar beam will reach the femoral neck, bounce off the neck and arrive back at the radar device. The or each radar device is arranged to be able to record the time taken for the beam to arrive back at the radar device. Accordingly, the reference location is the radial position of the radar device with respect to the femoral neck. From the information relating to how long the radar beam took to travel a distance equal to two times the distance from the reference location to the femoral neck, the distance between the reference location and the surface of the femoral neck can be established.

Infrared devices can be used to obtain data relating to the distance between a reference location and the surface of the femoral neck. Specifically, the or each infrared device may be arranged so as to be able to emit an infrared beam from a radial position with respect to the femoral neck, towards the femoral neck. The infrared beam will reach the femoral neck, bounce off the neck and arrive back at the infrared device. The or each infrared device is arranged to be able to record the time taken for the beam to arrive back at the infrared device. Accordingly, the reference location is the radial position of the infrared device with respect to the femoral neck. From the information relating to how long the infrared beam took to travel a distance equal to two times the distance from the reference location to the femoral neck, the distance between the reference location and the surface of the femoral neck can be established.

Ultrasound devices can be used to obtain data relating to the distance between a reference location and the surface of the femoral neck. Specifically, the or each ultrasound device may be arranged so as to be able to emit an ultrasound wave from a radial position with respect to the femoral neck, towards the femoral neck. The ultrasound wave will reach the femoral neck, bounce off the neck and arrive back at the ultrasound device. The or each ultrasound device is arranged to be able to record the time taken for the ultrasound wave to arrive back at the ultrasound device. Accordingly, the reference location is the radial position of the ultrasound device with respect to the femoral neck. From the information relating to how long the ultrasound wave took to travel a distance equal to two times the distance from the reference location to the femoral neck, the distance between the reference location and the surface of the femoral neck can be established.

Electromagnetic devices can be used to obtain data relating to the distance between a reference location and the surface of the femoral neck. Specifically, the or each electromagnetic device may be arranged so as to be able to emit an electromagnetic wave from a radial position with respect to the femoral neck, towards the femoral neck. The electromagnetic wave will reach the femoral neck, bounce off the neck and arrive back at the electromagnetic device. The or each electromagnetic device is arranged to be able to record the time taken for the electromagnetic wave to arrive back at the electromagnetic device. Accordingly, the reference location is the radial position of the electromagnetic device with respect to the femoral neck. From the information relating to how long the electromagnetic wave took to travel a distance equal to two times the distance from the reference location to the femoral neck, the distance between the reference location and the surface of the femoral neck can be established.

While several sensor devices are described in this disclosure, it will be appreciated by one skilled in the art that any other suitable sensor device could also be used.

In another embodiment, the targeting device comprises one or more retractable arms, each of which can move from a retracted state to an extended state. In such an embodiment, there may be two or more retractable arms, three or more, four or more, or any number of retractable arms as needed. The retractable arms may be extended and retracted by any suitable system, for example the retractable arms may be moveable by an electrometrical system. In particular, this system may comprise combinations of known motors and electronic devices that permit the arms to be extended and retracted. The extension and retraction of the arms may be controlled by any number of mechanisms known in the art. In one embodiment, the retractable arms are connected to the computer device and software, and the extension and retraction of the arms is controlled by the software. In another embodiment, the extension and retraction of the arms may be controlled by a person.

The one or more sensor devices may be located on the one or more retractable arms. In particular, the sensor device or devices may each be located on the distal end of a retractable arm. Each retractable arm may be provided with one or more sensor device. Preferably, there are the same number of arms as sensor devices, and a single sensor device is provided on each arm.

Movement of the one or more retractable arms from the retracted state to the extended state can move the one or more sensor devices from a position outside the body to a position inside the body, over the femoral neck and radially spaced therefrom.

The one or more retractable arms may be any suitable size and shape. The one or more retractable arms may be a curved shape, e.g. an arc shape. This allows the one or more sensor devices to be readily moved over the femoral head, when present, and to a position located over the femoral neck. The one or more retractable arms may suitably be rigid, or they may be flexible.

The targeting device may further comprise a body portion, on which the retractable arms may be mounted. Preferably, the retractable arms are mounted at one end of the body portion.

The use of one or more retractable arms, with the sensor devices provided on the arms, is advantageous as it minimizes the amount of equipment that needs to be placed inside the body. In particular, only the arms and sensor devices need enter the body; the remainder of the device can remain outside the body throughout the procedure.

In one embodiment, the targeting device comprises a sleeve component, comprising a first wall having an exterior surface and an interior surface that defines an inner bore, the wall running from a proximal end of the sleeve component to a distal end of the sleeve component, the sleeve component being shaped and sized such that it can be placed over the femoral neck with some or all of the femoral neck located within the bore. The one or more sensor devices can be located in the sleeve component.

The sleeve component can be moved from a position outside the body, to a position inside the body, with some or all of the femoral neck located within the bore of the sleeve component. The one or more sensor devices can thus be moved from a position outside the body to a position inside the body, over the femoral neck and radially spaced therefrom. The sleeve component is shaped and sized such that it can be placed over the femoral neck with some or all of the femoral neck located within its bore.

The first wall of the sleeve component may form a cylinder shape. Alternatively, the first wall of the sleeve component may form a hollow square prism shape, a hollow rectangular prism shape, a hollow hexagonal prism shape, or a hollow octagonal prism shape. The first wall of the sleeve component could also be any other regular or irregular shape, provide that it defines a bore within which some or all of the femoral neck can be located.

Suitably, the bore of the sleeve component has a diameter of 2.5 cm or more, such as from 3 cm to 8 cm, preferably from 3 cm to 7 cm, for example from 3.5 cm to 6.5 cm, more preferably from 4 cm to 6.5 cm.

The sleeve component preferably has a length such that it can be placed over the femoral head down to the base of the femoral neck. The sleeve component may suitably have a length of 3 cm or more, such as from 3.5 cm to 7 cm, preferably from 4 cm to 6 cm.

In one embodiment, the sleeve component is provided with a second wall, with the second wall being located outside of the first wall and being shaped so as to define a cavity between the first wall and the second wall.

In one embodiment, the second wall is of a substantially corresponding shape to the first wall, so that the cavity between the first and second wall is of a substantially constant depth. In an alternative embodiment, the second wall is of a different shape to the first wall.

The cavity formed between the first and second walls may be any suitable depth. Preferably, the cavity is large enough that it can house some or all of the sensor devices. However, it is clearly preferred that the depth of the cavity, and hence the overall diameter of the device, is kept as small as possible while achieving the desired function, in order to make the device more readily useable during surgery. Accordingly, it is preferred that the depth of the cavity between the first and second walls is 2 cm or less, more preferably 1.5 cm or less, most preferably 1 cm or less.

The distal end of the sleeve component is open so that it can be placed over the femoral head/femoral neck. The proximal end of the sleeve component may be open or may be closed apart from the guide sleeve holder.

The guide sleeve may be any suitable shape and size for receiving a guide wire in its bore. Suitably, the guide sleeve may be an elongate cylindrical shape with a bore suitable for receiving a guide wire. The guide sleeve may in particular be a conventional guide sleeve for a guide wire as known in the art.

The guide sleeve location adjustor may be electrometrical. In particular, it may comprise combinations of known motors and electronic devices that permit the guide sleeve to be moved in the anterior, posterior, medial and lateral directions. In one embodiment, the guide sleeve location adjustor is connected to the computer device and software, and the movement of the guide sleeve is controlled by the software. In an alternative embodiment, the guide sleeve location adjustor is controlled by a person.

The software may be any software capable of receiving and processing the data from the or each sensor device, so as to determine the position of the femoral neck centerline. It will be appreciated by one skilled in the art that the exact nature of the software will vary depending upon the sensor device used. The software should also be able to process the data recorded by the sensor device and from this determine the position of the centerline of the femoral neck. In one embodiment, the software is such that, having determined the location of the centerline of the femoral neck, it controls the guide sleeve location adjustor and causes the adjustor to move the guide sleeve in line with this calculated centerline position. In an alternative embodiment, the targeting device further comprises a screen and the software is such that, having determined the location of the centerline of the femoral neck, it provides an image of the femoral neck and its centerline location on the screen so that a person can manually use the guide sleeve location adjustor to move the guide sleeve in line with this calculated centerline position.

It will be appreciated by one skilled in the art that the targeting device can be made from any suitable material, such as metal or plastics material. Those portions of the device that are intended to enter into the body should be made from surgically acceptable material.

A method of locating the centerline of the osteotomised base of the femoral neck at the head-neck junction with a targeting device in accordance with any of the described embodiments may comprise the steps of moving the or each sensor device to a position inside the body, over the femoral neck and radially spaced therefrom; using the or each sensor device to obtain data relating to the distance between the sensor device and the surface of the femoral neck in at least one location in the anterior-posterior plane and at least one location in the superior-inferior plane; using software to determine the position of the centerline of the femoral neck based on the data received from the or each sensor device; and using the guide sleeve location adjustor to move the guide sleeve to be in line with the determined centerline. In an additional embodiment, the method may further comprise the step of passing a guide wire through the guide sleeve to mark the centerline of the osteotomised femoral neck.

A method of locating the centerline through the femoral neck and femoral head with a targeting device in accordance with any of the described embodiments may comprise the steps of moving the or each sensor device to a position inside the body, over the femoral neck and radially spaced therefrom; using the or each sensor device to obtain data relating to the distance between the sensor device and the surface of the femoral neck in at least one location in the anterior-posterior plane and at least one location in the superior-inferior plane; using the software to determine the position of the centerline of the femoral head and neck based on the data received from the or each sensor device; and using the guide sleeve location adjustor to move the guide sleeve to be in line with the determined centerline. In an additional embodiment, the method further comprises the step of passing a guide wire through the guide sleeve to mark the centerline of the femoral head and neck.

Reference will now be made to the embodiments illustrated in the drawings and specific language will be used to describe the same. It will nevertheless be understood that no limitation of the scope of this disclosure is thereby intended by reference to these figures.

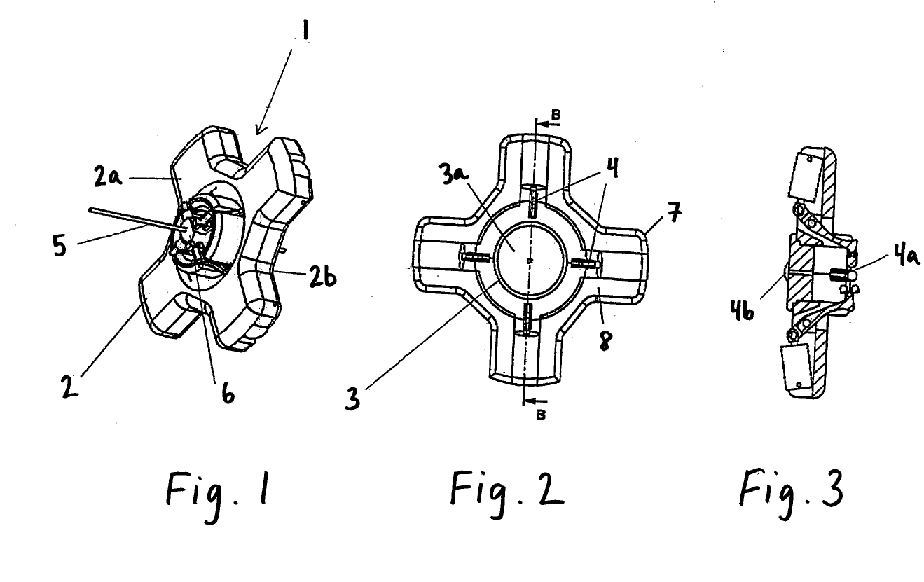

FIGS. 1-3 show a first targeting device 1 suitable for use in open or minimally invasive hip replacement surgery. The device 1 comprises a sleeve component 2 having a proximal end 2a and a distal end 2b. The sleeve component 2 comprises a cylindrical shaped inner wall 3. The inner wall 3 has an interior surface that defines an inner bore 3a. The sleeve component 2 is sized in view of the femoral neck in question, and can be placed over the femoral neck with some or all of the femoral neck located within the bore 3a, with the distal end 2b of the sleeve component 2 towards the base of the femoral neck. The sleeve component also has an outer wall 7. The outer wall is in the shape of a cross. The outer wall 7 and inner wall 3 together define a cavity 8.

The device 1 also comprises four sensor devices 4 for obtaining data relating to the location of the surface of the femoral neck with respect to a reference position when some or all of the femoral neck is located within the bore of the sleeve component. Two of the devices are located to obtain data in the anterior-posterior plane and two of the devices are located to obtain data in the superior-inferior plane. The devices 4 are angularly spaced apart by 90°. The devices 4 are located within cavity 8 of the sleeve component 2.

The sensor devices 4 are electromechanical devices. Each electromechanical device comprises an electronically driven motor 4a with an end probe 4b that is able to move in a straight line from its neutral position to a position abutting the surface of the femoral neck. Each electromechanical device is arranged to be able to record the distance moved by the electronically driven motor and probe from the neutral position to the position abutting the surface of the femoral neck.

The device 1 further comprises a guide sleeve 5. The guide sleeve 5 is located at the proximal end 2a of the sleeve component 2. The guide sleeve is an elongate cylindrical shape with a bore capable of receiving a guide wire.

The device 1 additionally comprises guide sleeve location adjustor 6 suitable for moving the guide sleeve in the anterior, posterior, medial and lateral directions. The guide sleeve location adjustor 6 is electromechanical, comprising a combination of known motors and electronic devices to allow movement of the guide sleeve in the anterior, posterior, medial and lateral directions.

The device 1 also comprises a computer device and software (not shown) for receiving data from the sensor devices 4 and processing this data so as to determine the location of the centerline of the femoral neck. The software controls the guide sleeve location adjustor 6, causing it to move the guide sleeve in the anterior, posterior, medial and lateral directions as necessary so as to be in line with the determined centerline of the femoral neck.

In use, targeting device 1 permits location of the central longitudinal axis of the femoral neck. In minimally invasive hip replacement surgery (as described in PCT Application Publication No. WO 03/065906 of the same inventor, which is hereby incorporated by reference) or in open hip replacement surgery, the following steps may be carried out: (1) providing a targeting device 1; (2) placing the sleeve component 2 over the femoral neck, so that some or all of the femoral neck is located within the bore 3a of the sleeve component, with the distal end 2b located towards the base of the femoral neck, such that the sensor devices 4 are placed over the femoral neck and radially spaced therefrom; (3) moving each electronically driven motor 4a with an end probe 4b in a straight line from its neutral position to a position abutting the surface of the femoral neck, and recording the distance moved; (4) using the software to determine the position of the centerline of the femoral head and neck based on the data received from each sensor device 4; and (5) using the guide sleeve location adjustor 6 to move the guide sleeve 5 to be in line with the determined centerline.

A guide wire can then be passed through the guide sleeve 5 to mark the centerline of the femoral neck. The femoral neck can then be reamed accurately and centrally and the resultant prosthesis will be accurately and centrally positioned.

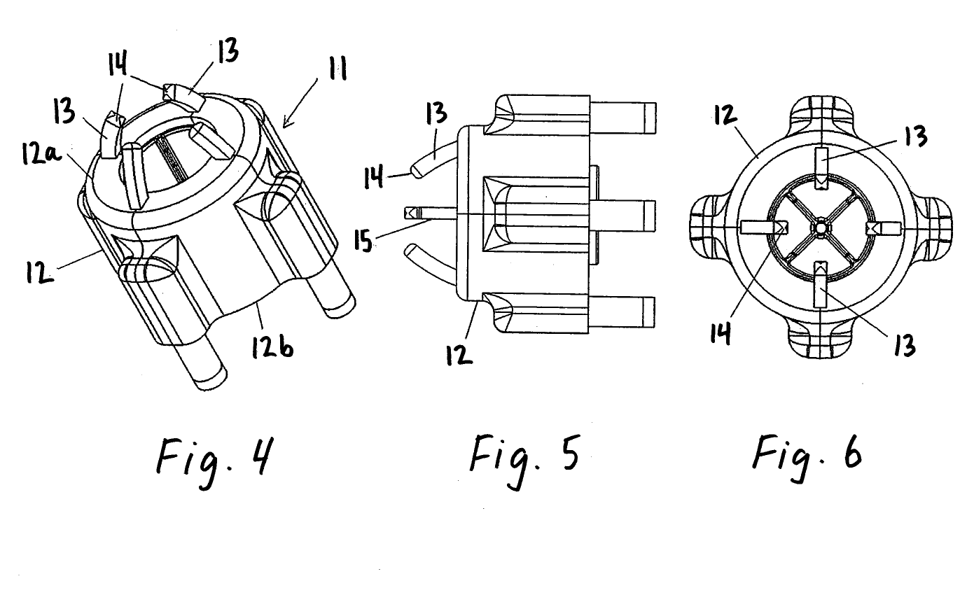

FIGS. 4-6 show a second targeting device 11 suitable for use in open or minimally invasive hip replacement surgery. The device 11 comprises a main body 12 which is substantially cylindrical in shape and has a proximal end 12a and a distal end 12b. The device 11 comprises four retractable arms 13 located at the proximal end 12a of the main body. The arms 13 are angularly spaced apart by 90°. The arms 13 can extend from their retracted position to an extended position. The arms are curved.

The device 11 also comprises four sensor devices 14 for obtaining data relating to the location of the surface of the femoral neck with respect to a reference position when some or all of the femoral neck is located within the bore of the sleeve component. Two of the devices are located to obtain data in the anterior-posterior plane and two of the devices are located to obtain data in the superior-inferior plane. The sensor devices 14 are located at the distal end of the retractable arms 13.

In this embodiment, the sensor devices 14 are electromechanical devices. Each electromechanical device comprises an electronically driven motor with an end probe that is able to move in a straight line from its neutral position to a position abutting the surface of the femoral neck. Each electromechanical device is arranged to be able to record the distance moved by the electronically driven motor and probe from the neutral position to the position abutting the surface of the femoral neck.

The device 11 further comprises a guide sleeve 15. The guide sleeve 15 is located at the proximal end 12a of the main body 12. The guide sleeve is an elongate cylindrical shape with a bore capable of receiving a guide wire.

The device 11 additionally comprises guide sleeve location adjustor (not shown) suitable for moving the guide sleeve in the anterior, posterior, medial and lateral directions. The guide sleeve location adjustor is electromechanical, comprising a combination of known motors and electronic devices to allow movement of the guide sleeve in the anterior, posterior, medial and lateral directions.

The device 11 also comprises a computer device and software (not shown) for receiving data from the sensor devices 14 and processing this data so as to determine the location of the centerline of the femoral neck. The software controls the guide sleeve location adjustor, causing it to move the guide sleeve in the anterior, posterior, medial and lateral directions as necessary so as to be in line with the determined centerline of the femoral neck.

In use, targeting device 11 of the present disclosure permits location of the central longitudinal axis of the femoral neck. In minimally invasive hip replacement surgery (as described in PCT Application Publication No. WO 03/065906 of the same inventor, which is hereby incorporated by reference) or in open hip replacement surgery, the following steps may be carried out: (1) providing a targeting device 11; (b) extending each retractable arm 13 from its retracted position to its extended position, so as to move each electromechanical sensor device 14 from a position outside the body to a position inside the body, over the femoral neck; (c) moving each electromechanical sensor device 14 in a straight line from its neutral position to a position abutting the surface of the femoral neck, and recording the distance moved; (d) using the software to determine the position of the centerline of the femoral head and neck based on the data received from each sensor device 14; and (5) using the guide sleeve location adjustor to move the guide sleeve 15 to be in line with the determined centerline.

A guide wire can then be passed through the guide sleeve 15 to mark the centerline of the femoral neck. The femoral neck can then be reamed accurately and centrally and the resultant prosthesis will be accurately and centrally positioned.

Although the devices 1, 11 have been illustrated in relation to the use of electromechanical sensor devices, it will be understood by one of ordinary skill in the art that other sensor devices, in particular radar, infrared, ultrasound or electromagnetic sensor devices, could equally be used.

In view of the many possible embodiments to which the principles of this disclosure may be applied, it should be recognized that the detailed embodiments are illustrative only and should not be taken as limiting the scope of the disclosure. The present disclosure can be further modified within the scope and spirit of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

1. A targeting device for use in hip surgery to allow the position of the centerline of the femoral neck to be located, the device comprising:
one or more sensor devices, moveable to a position inside the body that is over the femoral neck and radially spaced therefrom, the device or devices being suitable for obtaining data relating to the location of the surface of the femoral neck with respect to a reference position, in both the anterior-posterior and superior-inferior planes;
a guide sleeve, having a bore capable of receiving a guide wire;
a guide sleeve location adjustor, suitable for moving the guide sleeve in the anterior, posterior, medial and lateral directions; and
a computer device with software for receiving data from the sensor device or devices and processing this data so as to determine the location of the centerline of the femoral neck.

2. The device of claim 1 wherein there are one or more sensor devices that are moveable so as to obtain data at two or more different radial locations around the femoral neck, with at least one location being such that data is obtained in the anterior-posterior plane and at least one location being such that data is obtained in the superior-inferior plane.

3. The device of claim 2 wherein the one or more sensor devices are moveable so as to obtain data at four or more different radial locations around the femoral neck.

4. The device of claim 2 wherein the one or more sensor devices are moveable to two or more different radial locations where data is obtained in the superior-inferior plane.

5. The device of claim 2 wherein the one or more sensor devices are moveable to two or more different radial locations where data is obtained in the anterior-posterior plane.

6. The device of claim 1 wherein there are two or more angularly spaced sensor devices, with at least one device being able to obtain data in the anterior-posterior plane and at least one device being able to obtain data in the superior-inferior plane.

7. The device of claim 6 wherein there are four or more angularly spaced sensor devices.

8. The device of claim 6 wherein there are two or more devices able to obtain data in the superior-inferior plane.

9. The device of claim 6 wherein there are two or more devices able to obtain data in the anterior-posterior plane.

10. The device of claim 1 wherein the sensor devices are selected from electro-mechanical devices, radar devices, infrared devices, ultrasound devices and electromagnetic devices.

11. The device of claim 1 wherein the targeting device comprises:
one or more retractable arm, each of which can move from a retracted state to an extended state.

12. The device of claim 11 wherein there are two or more retractable arms.

13. The device of claim 11 wherein the one or more sensor devices are located on the one or more retractable arms.

14. The device of claim 11 wherein there are the same number of arms as sensor devices, and a single sensor device is provided on each arm.

15. The device of claim 11 wherein the one or more retractable arms are a curved shape.

16. The device of claim 11 wherein the one or more retractable arms are rigid.

17. The device of claim 1 wherein the targeting device comprises:
a sleeve component, comprising a first wall having an exterior surface and an interior surface that defines an inner bore, the wall running from a proximal end of the sleeve component to a distal end of the sleeve component, the sleeve component being shaped and sized such that it can be placed over the femoral neck with some or all of the femoral neck located within the bore.

18. The device of claim 17 wherein the one or more sensor devices are located in the sleeve component.

19. The device of claim 17 wherein the first wall of the sleeve component forms a cylinder shape.

20. A method of locating the centerline of the osteotomised base of the femoral neck at the head-neck junction, which method comprises:
(a) providing a targeting device, for use in hip surgery to allow the position of the centerline of the femoral neck to be located, the device comprising:
one or more sensor device, moveable to a position inside the body that is over the femoral neck and radially spaced therefrom, the device or devices being suitable for obtaining data relating to the location of the surface of the femoral neck with respect to a reference position, in both the anterior-posterior and superior-inferior planes;
a guide sleeve, having a bore capable of receiving a guide wire;
a guide sleeve location adjustor, suitable for moving the guide sleeve in the anterior, posterior, medial and lateral directions; and
a computer device with software for receiving data from the sensor device or devices and processing this data so as to determine the location of the centerline of the femoral neck;
(b) moving the or each sensor device to a position inside the body, over the femoral neck and radially spaced therefrom;
(c) using the or each sensor device to obtain data relating to the distance between the sensor device and the surface of the femoral neck in at least one location in the anterior-posterior plane and at least one location in the superior-inferior plane;
(d) using the software to determine the position of the centerline of the femoral neck based on the data received from the or each sensor device; and
(e) using the guide sleeve location adjustor to move the guide sleeve to be in line with the determined centerline.

21. A method of locating the centerline through the femoral neck and femoral head, which method comprises:
(a) providing a targeting device for use in hip surgery to allow the position of the centerline of the femoral neck to be located, the device comprising:
one or more sensor device, moveable to a position inside the body that is over the femoral neck and radially spaced therefrom, the device or devices being suitable for obtaining data relating to the location of the surface of the femoral neck with respect to a reference position, in both the anterior-posterior and superior-inferior planes;
a guide sleeve, having a bore capable of receiving a guide wire;
a guide sleeve location adjustor, suitable for moving the guide sleeve in the anterior, posterior, medial and lateral directions; and
a computer device with software for receiving data from the sensor device or devices and processing this data so as to determine the location of the centerline of the femoral neck;
(b) moving the or each sensor device to a position inside the body, over the femoral neck and radially spaced therefrom;
(c) using the or each sensor device to obtain data relating to the distance between the sensor device and the surface of the femoral neck in at least one location in the anterior-posterior plane and at least one location in the superior-inferior plane;
(d) using the software to determine the position of the centerline of the femoral head and neck based on the data received from the or each sensor device; and
(e) using the guide sleeve location adjustor to move the guide sleeve to be in line with the determined centerline.
